(19) United States
(12) Patent Application Publication
(10) Pub. No.: US 2010/0217059 A1
(43) Pub. Date: Aug. 26, 2010

(54) Process for Recovering Ethylene From an Autothermal Cracking Reactor Effluent

(76) Inventors: Rian Reyneke, Katy, TX (US); Michael J. Foral, Aurora, IL (US); Graeme Parker, Falkirk (GB); Wayne W.Y. Eng, League, TX (US)

Correspondence Address: NIXON & VANDERHYE, PC, 901 NORTH GLEBE ROAD, 11TH FLOOR, ARLINGTON, VA, 22203, US

(21) Appl. No.: 11/922,028

(22) PCT Filed: Jul. 28, 2005
(86) PCT No.: PCT/US2005/026984

§ 371 Date: Dec. 12, 2007

(51) Int. Cl. C07C 4/06

(52) U.S. Cl. 585/651

(57) ABSTRACT

The process of this invention represents an improved, low-energy method for recovering a purified ethylene product from the effluent of an autothermal cracking reactor. The process consists of a cracked gas chilling train, a front-end ethylene distributor, a demethanizer, and a C2 splitter. Hydrocarbons heavier than ethylene, including ethane, propylene, and propane are recycled in a single stream to the ATC reactor. Acetylene removal from the ethylene product can be accomplished either through a front-end hydrogenation unit or an acetylene extraction unit. This invention is particularly useful when the fresh hydrocarbon feed to the autothermal cracking reactor is ethane or a mixture of ethane and propane.

### STATEMENT REGARDING FEDERALLY SPONSORED RESEARCH OR DEVELOPMENT

This invention was made with government support under United States Department of Energy Cooperative Agreement No. DE-FC07-01ID 14090.

### PROCESS

1. Field of the Invention

This invention relates to the recovery of ethylene from the effluent of an autothermal cracking reactor utilizing an ethylene distributor.

2. Background

The production of ethylene (and other olefins) from a paraffinic hydrocarbon feed by steam cracking is well-known in the art. In a typical steam cracking process a paraffinic hydrocarbon feed such as ethane, propane, butane, naphtha, gas oil, or a combination of these feeds is introduced, along with a controlled amount of steam, into a cracking furnace, in which the steam/paraffinic hydrocarbon mixture is heated to high temperatures and the paraffinic hydrocarbon feed is thermally cracked to yield a range of products.

This cracking reaction typically takes place under relatively low pressure, usually below 25 psig.

The cracked gas composition depends on a number of factors, including the composition of the paraffinic hydrocarbon feed and the cracking furnace operating conditions, but generally comprises hydrogen, methane, ethylene, ethane, propylene, propane and higher hydrocarbons and water vapor. Small amounts of other products, such as acetylene, propadiene, methyl acetylene, carbon monoxide and carbon dioxide may also be present.

The ethylene and any other desired products must thus be separated from the cracked gas.

Typically, the hot cracked gas mixture from the cracking furnace is first cooled, for example by directing it to a transfer line exchanger in which it is cooled by raising high pressure steam. Further cooling can be achieved by various means, including boiler feed water preheating, oil quenching, and water quenching.

The quenched cracked gas may then be compressed to relatively high pressure, typically around 500 psig, before carbon dioxide and any remaining water is removed and the compressed, dried cracked gas mixture is directed to the separation section of the plant.

There are many methods by which the compounds in the cracked gas can be separated to yield olefin product(s), fuel gas, hydrogen, recycle streams, and heavy products. Some suitable methods are described in Hydrocarbon Processing, March 2003, pp. 96-99.

In general, however, the separation methods typically fall into one of three types.

First are the front-end demethanizer methods, in which the first step of separation is to chill the cracked gas and remove the methane and lighter components from the remaining heavier hydrocarbons.

Front-end deethanizer methods chill the cracked gas and then remove the ethane and lighter components from the remaining heavier components.

Front-end depropanizer flowsheets are similar, except that the first step is to remove the propane and lighter components from the heavier hydrocarbons.

The optimal separation method will depend on many factors, including feed type, product requirements, energy cost, and feed cost, among others.

An alternative method of production of olefins from a paraffinic hydrocarbon feed is autothermal cracking, as described for example, in EP 332289B; EP-529793B; EP-A-0709446 and WO 00/14035. In an autothermal cracking process a paraffinic hydrocarbon-containing feedstock is mixed with oxygen and passed over an autothermal cracking catalyst. The autothermal cracking catalyst is capable of supporting combustion beyond the fuel rich limit of flammability. Combustion of a portion of the feed is initiated on the catalyst surface and the heat required to raise the reactants to the process temperature and to carry out the endothermic cracking process is generated in situ.

More recently, it has been found that autothermal cracking processes can tolerate significant levels of unsaturated compounds in the feed to the process, as described in WO 2004/087626.

In contrast, for steam cracking it is well-known that unsaturated hydrocarbons have a high propensity to cause carbonaceous fouling of the furnace process equipment. Thus, it is undesirable to recycle high levels of olefinic materials to a conventional steam cracker (low levels of unsaturated hydrocarbons are inherently present in many hydrocarbon feeds, and it is undesirable to add to these).

Thus, whilst conventional ethylene recovery and purification (separation) methods have included steps to ensure that olefinic materials are largely removed from any stream that is to be recycled to the steam cracker, we have found that the steps required to recover and purify an ethylene product from the cracked product from an autothermal cracking process may be operated with significantly reduced energy requirement by allowing olefinic materials to be present in the hydrocarbon recycle streams to an autothermal cracking reactor. This energy reduction allows the autothermal cracking-based manufacture of ethylene to be achieved in a more economic manner, and in particular provides a reduced refrigeration duty for the separations. Overall yields of ethylene may also be improved by the recycle of unsaturated hydrocarbons other than ethylene, since these may be expected to preferentially combust (in preference to the paraffinic hydrocarbon feed) in the autothermal cracking reaction.

Thus, in a first aspect, the present invention provides a process for the production of ethylene from a paraffinic hydrocarbon-containing feed, said process comprising the steps of:
(i) autothermally cracking the paraffinic hydrocarbon-containing feed with a molecular oxygen-containing gas in contact with a catalyst capable of supporting combustion beyond the normal fuel rich limit of flammability to produce a product stream comprising ethylene, ethane, propylene, methane, carbon monoxide, hydrogen and hydrocarbons heavier than propylene,
(ii) chilling at least a portion of said product stream and, without first passing said chilled product stream through a deethanizer, passing the chilled product stream to a first separations step comprising one or more distillation columns, one of which operates as an ethylene distributor column, to recover a light gas stream comprising methane, carbon monoxide, hydrogen and less than 5% of the ethylene contained in said chilled product stream, a first liquid stream comprising ethylene, ethane, propylene and hydrocarbons heavier than propylene and substantially free of components lighter than ethylene, and a first ethylene-rich product stream,
(iii) directing said first liquid stream to a second separations step comprising a C2 splitter distillation column, to recover a second ethylene-rich product stream, a product stream comprising ethane, propylene, and hydrocarbons heavier than propylene,
(iv) recycling said product stream comprising ethane, propylene, and hydrocarbons heavier than propylene to the autothermal cracking reactor of step (i), and
(v) recovering from said first ethylene-rich product stream a first purified ethylene product,
(vi) recovering from said second ethylene-rich product stream a second purified ethylene product.

In step (i) of the process of the present invention, a paraffinic hydrocarbon-containing feed is autothermally cracked with a molecular oxygen-containing gas in contact with a catalyst capable of supporting combustion beyond the normal fuel rich limit of flammability to produce a product stream comprising ethylene, ethane, propylene, carbon monoxide, hydrogen and hydrocarbons heavier than propylene.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

The paraffinic hydrocarbon-containing feed may comprise liquid or gaseous paraffinic hydrocarbons. Suitable liquid feeds include naphtha, gas oils, vacuum gas oils and mixtures thereof. Preferably, however, gaseous feeds such as ethane, propane, butane and mixtures thereof are employed.

The paraffinic hydrocarbon-containing feedstock may be fed with any suitable molecular oxygen-containing gas. Suitably, the molecular oxygen-containing gas is molecular oxygen itself, air and/or mixtures thereof. The molecular oxygen-containing gas may be mixed with an inert gas such as nitrogen or argon.

Preferably the paraffinic hydrocarbon-containing feedstock and molecular oxygen-containing gas are fed to the autothermal cracker at a ratio of paraffinic hydrocarbon to molecular oxygen of 5 to 16 times, preferably 5 to 13.5 times, more preferably 6 to 10 times, the stoichiometric ratio of paraffinic hydrocarbon to molecular oxygen required for complete combustion of the hydrocarbon to carbon dioxide and water.

The paraffinic hydrocarbon is passed over the catalyst at a gas hourly space velocity of greater than 10,000 h −1, preferably above 20,000 h −1 and most preferably, greater than 100,000 h −1. It will be understood, however, that the optimum gas hourly space velocity will depend upon the pressure and nature of the feed composition.

Additional feed components may be co-fed into the autothermal cracker, such as hydrogen, carbon monoxide, carbon dioxide or steam.

The autothermal cracking process is suitably carried out at a catalyst exit temperature in the range 600° C. to 1200° C., preferably, in the range 850° C. to 1050° C. and, most preferably, in the range 900° C. to 1000° C. To avoid further reactions taking place, the autothermal cracking reactor product stream should be rapidly cooled, typically by cooling to between 750-600° C. within 20 milliseconds of formation. The autothermal cracking process may be carried out at any suitable pressure between 0 and 50 barg, preferably between 1 barg and 35 barg. Advantageously wherein the autothermal cracking process is operated at a pressure of greater than 20 barg the products are cooled to between 750-600° C. within 10 milliseconds of formation.

Other products, such as acetylene, water vapor and carbon dioxide may also be present, as may be unreacted oxygen, in the cracked gas stream initially recovered from the autothermal cracking reaction. Prior to passing said stream as a product stream comprising ethylene, ethane, propylene, methane, carbon monoxide, hydrogen and hydrocarbons heavier than propylene to the subsequent separation steps of the process of the present invention, the cracked gas stream from the autothermal cracker is first treated to remove any carbon dioxide, water and unreacted molecular oxygen.

Carbon dioxide is removed, for example, using an amine-based absorption system such as MEA or TEA (or mixtures of both), or other commercially available CO2 removal process.

Any residual oxygen may be removed, for example, by contact with a suitable catalyst. Residual water may be removed next. Any suitable drying process may be used, for example by use of a suitable molecular sieve.

All or a portion of this autothermal cracking reactor product stream (after carbon dioxide removal, oxygen removal and/or water removal) may then be passed as the product stream comprising ethylene, ethane, propylene, carbon monoxide, hydrogen, and hydrocarbons heavier than propylene to the subsequent separation steps of the first aspect of the present invention.

It may also be desirable to remove all or a portion of any acetylene in the product stream prior to the subsequent separation steps. For such removal a front-end hydrogenation system or other suitable means can be used. It should be noted that the presence of high concentrations of carbon monoxide in the product stream may inhibit the operation of a standard front-end acetylene hydrogenation system. The acetylene hydrogenation reaction may be sufficiently inhibited by the carbon monoxide that it is not possible to achieve product-purity ethylene through the use of front-end hydrogenation. Typically, the concentration of acetylene to ethylene in the purified ethylene product stream should be below about 5 ppm by weight. If front-end hydrogenation cannot be used to achieve this level of acetylene removal, then an alternate means of acetylene removal must be used within the process of this invention. Suitable examples are described further below.

In step (ii) of the process of the present invention, at least a portion of said product stream is chilled and subsequently passed to a first separations step comprising a distillation column which acts as an ethylene distributor column (this distillation column being referred to hereinafter as an ethylene distributor column), and optionally one or more further distillation columns. It is a feature of the present invention that said stream is not first passed through a deethanizer i.e. that it is not necessary to separate hydrocarbons heavier than ethane from the ethane, ethylene and lighter components of the product stream prior to passing the product stream to the ethylene distributor column.

For the purpose of this invention, an ethylene distributor column is one in which a sharp split is made between components lighter than ethylene and components heavier than ethylene. Therefore the ethylene distributor column produces an overhead or side stream containing ethylene and components lighter than ethylene, but few if any components heavier than ethylene. In particular, the ethylene distributor overhead or side stream contains a sufficiently low concentration of ethane that no further ethane/ethylene separation is needed in order to produce a purified ethylene product from this stream. Typically the molar ratio of ethane to ethylene in the ethylene distributor overhead or side stream is less than about 0.005, preferably less than about 0.001, though in some cases a molar ratio higher than 0.005 could be tolerated. The ethylene distributor bottoms stream contains ethylene and any components heavier than ethylene that enter the ethylene distributor.

Where one or more further distillation columns are present, energy savings can be gained by thermally coupling (also called recycle-coupling) pairs of distillation columns such that all or at least part of the stripping vapor or reflux liquid of one column is provided by a vapor or liquid side-draw from a downstream column. Examples of completely thermally coupled distributed distillation systems which utilize an ethylene distributor have been disclosed in Manley et al., U.S. Pat. Nos. 5,675,054 and 5,746,066. For example, in 5,675,054 Manley teaches the preferred embodiments of his design for various steam cracking furnace feedstocks. In particular he teaches that for the pyrolysis of ethane or ethane/propane mixtures a front-end deethanizer configuration is preferred, wherein the deethanizer overhead feeds a downstream ethylene distributor column. In the process of the present invention we have found, surprisingly, that for a process with an autothermal cracking reactor instead of a conventional steam cracking furnace, because of the ability of the autothermal cracking reactor to handle the recycle of olefins, a front-end ethylene distributor in the absence of a front-end deethanizer is preferred.

In particular, the present invention provides for recovery of ethylene in a manner which reduces the overall energy required for the necessary separations, particularly the energy required to provide refrigeration for the process, which provides for a more economic ethylene manufacturing process. Moreover, compared with the prior art ethylene distributor-based ethylene recovery processes the process of this invention utilizes fewer distillation columns and therefore is expected to be simpler to operate and cheaper to construct.

In step (ii) of the process of the present invention there is recovered a light gas stream comprising methane, carbon monoxide, hydrogen and less than 5% of the ethylene contained in said chilled product stream, a first liquid stream comprising ethylene, ethane, propylene and hydrocarbons heavier than propylene and substantially free of components lighter than ethylene, and a first ethylene-rich product stream.

A number of alternative configurations for the first separations step can be utilized.

For example, in one embodiment the ethylene distributor column is provided in conjunction with a demethanizer column, such that the overhead of the ethylene distributor, which comprises methane, carbon monoxide, hydrogen and ethylene but little ethane or heavier hydrocarbons is fed to the demethanizer column.

In particular, step (ii) of this embodiment may comprise the steps of:

(a1)) directing the chilled product stream to an ethylene distributor distillation column and recovering therefrom an ethylene distributor overhead stream comprising ethylene, methane, carbon monoxide, and hydrogen and substantially free of ethane, and an ethylene distributor bottoms stream comprising ethylene, ethane, propylene, and optionally components heavier than propylene and substantially free of components lighter than ethylene;
(b1) withdrawing the ethylene distributor bottoms stream as the first liquid stream of step (ii);
(c1) directing at least a portion of the ethylene distributor overhead stream to a demethanizer column and recovering therefrom a demethanizer overhead stream comprising methane, carbon monoxide and hydrogen and a demethanizer bottoms stream comprising ethylene and substantially free of components lighter than ethylene;
(d1)) recovering said light gas stream of step (ii) from the demethanizer overhead stream of step (c1); and
(e1) withdrawing the demethanizer bottoms stream as the first ethylene rich product stream of step (ii).

In this embodiment, the bottoms stream from the ethylene distributor contains ethylene, ethane, propylene and hydrocarbons heavier than propylene, and forms the first liquid stream of step (ii) of the present invention. The bottoms of the demethanizer column comprises ethylene, typically at least 90 mol % ethylene, and may also contain acetylene if this has not been previously removed. This stream forms the first ethylene-rich product stream of step (ii). The overhead of the demethanizer comprises methane, carbon monoxide and hydrogen, and less than 5 mol % ethylene and forms the light gas stream of step (ii).

If the demethanizer overhead vapor contains a sufficient amount of ethylene, it may be desirable to process it further, for example by chilling, rectification, or a combination of chilling and rectification, in order to recover at least a portion of the ethylene therein, the remainder forming the light gas stream of step (ii).

In this embodiment it is preferred to provide stripping vapor to both the ethylene distributor column and the demethanizer column through the use of conventional reboilers in which a portion of the bottoms liquid product is vaporized and directed to the bottom of the column.

It may also be desirable to thermally couple the ethylene distributor column and the demethanizer column of this embodiment by directing a liquid sidestream from the demethanizer column to the top of the ethylene distributor column as reflux liquid. The liquid sidestream from the demethanizer column would typically be taken from a location near that where the ethylene distributor overhead stream enters the demethanizer.

Alternatively, the reflux liquid for the ethylene distributor column may be provided through partial condensation of the gross ethylene distributor overhead vapor stream. The chilling duty for said partial condensation is preferably provided by a closed-loop mixed refrigeration system.

In a second embodiment of step (ii) the ethylene distributor column is connected to a sidestripper column such that a sidestream is taken from the ethylene distributor column and passed to the sidestripper. The overhead from the side stripper is returned to the ethylene distributor column, typically near, and preferably just below, the location at which the sidestream was withdrawn.

In particular, step (ii) of this embodiment may comprise the steps of:

(a2) directing the entire chilled product stream to an ethylene distributor distillation column and recovering therefrom an ethylene distributor column overhead stream comprising methane, carbon monoxide, hydrogen and less than 5% of the ethylene contained in said chilled product stream, and an ethylene distributor column bottoms stream comprising ethylene, ethane, propylene, and hydrocarbons heavier than propylene and substantially free of components lighter than ethylene;
(b2) recovering said light gas stream of step (ii) from the ethylene distributor column overhead stream of step (a2);
(c2) withdrawing the ethylene distributor column bottoms stream as the first liquid stream of step (ii);
(d2) withdrawing a liquid sidedraw stream from the ethylene distributor column at a point intermediate between the top of the ethylene distributor column and the point where the chilled product stream enters the ethylene distributor column, wherein the liquid sidedraw stream comprises ethylene and methane, and is substantially free of ethane;
(e2) directing the liquid sidedraw stream to the top of a sidestripper column and recovering therefrom a sidestripper bottoms stream comprising ethylene and substantially free of components lighter than ethylene, and a sidestripper overhead stream comprising methane;
(f2) withdrawing the sidestripper bottoms stream as the first ethylene-rich product stream of step (ii), and
(g2) directing the sidestripper overhead stream to the ethylene distributor column of step (a2).

The ethylene distributor column combines the functions of the ethylene distributor and the demethanizer enriching section, that is, the section of the demethanizer column of the first embodiment which is above the feed stage, and the sidestripper column serves the function of the demethanizer stripper section, that is, the section of the demethanizer column of the first embodiment which is below the feed stage.

In this second embodiment, the bottoms stream from the ethylene distributor comprises ethylene, ethane, propylene and hydrocarbons heavier than propylene, and forms the first liquid stream of step (ii) of the present invention. The overhead of the ethylene distributor column comprises methane, carbon monoxide and hydrogen, and less than 5 mol % ethylene and forms the light gas stream of step (ii). If the ethylene distributor overhead vapor contains a sufficient amount of ethylene, it may be desirable to process it further, for example by chilling, rectification, or a combination of chilling and rectification, in order to recover at least a portion of the ethylene therein, as described for the first embodiment.

The bottoms of the sidestripper column comprises ethylene, typically at least 90 mol % ethylene, and may also contain acetylene if this has not been previously removed. This stream forms the first ethylene-rich stream of step (ii).

In this second embodiment it is preferred to provide stripping vapor to both the ethylene distributor column and the sidestripper column through the use of conventional reboilers in which a portion of the bottoms liquid is vaporized and directed to the bottom of the column.

In a third embodiment of step (ii) the ethylene distributor column is a divided wall column. In effect, the ethylene distributor column and sidestripper column of the second embodiment are combined into a single divided wall column. In the divided wall column a dividing wall extends from a middle point of the column all the way to the bottom of the column, thereby creating a single rectifying section above the wall and two half-sections on either side of the wall. The chilled product stream of step (ii) enters the divided wall column in one of the half-sections, at a location below the top of the wall. The half-section which the feed enters, along with the single rectifying section, provide the function of the ethylene distributor column of the second embodiment. The other (non-feed) half-section of the divided wall column serves the function of the sidestripper column of the second embodiment. The design of this divided wall column is similar to that of published U.S. patent application US 2004/4182751, although the function is different. The divided wall column of the third embodiment is operated such that the vapor exiting from the feed half-section contains ethylene but is essentially free of ethane when it reaches the top of the dividing wall.

The overhead stream recovered from the ethylene distributor dividing wall column comprises primarily methane, carbon monoxide and hydrogen and forms the light gas stream of step (ii). If the stream contains a sufficient amount of ethylene, it may be desirable to process it further, for example by chilling, rectification, or a combination of chilling and rectification, in order to recover at least a portion of the ethylene, as described for the first embodiment above.

The bottoms product from the feed half-section forms the first liquid stream of step (ii) and the bottoms product from the other (sidestripper) half-section forms the first ethylene-rich product stream of step (ii).

In this embodiment it is preferred to provide stripping vapor to both of the half-sections of the divided wall column through the use of conventional reboilers in which a portion of the bottoms liquid product from each side of the dividing wall is vaporized and directed to the bottom of the respective half-section of the column. Therefore the divided wall column will employ at least two reboilers, at least one arranged on each side of the dividing wall.

In step (iii) of the process of this invention the first liquid stream is directed to a C2 splitter column. It may be desirable to remove acetylene from the first liquid stream before directing it to the C2 splitter column. In this case a conventional back-end acetylene hydrogenation system may be used in which the stream is contacted with a suitable catalyst in the presence of hydrogen to convert an effective amount of the acetylene to ethylene and/or ethane.

An ethylene-rich product, which forms the second ethylene-rich product stream of step (iii), is withdrawn from an upper section of the C2 splitter column, either as a vapor or liquid overhead product or as a liquid sidestream at the bottom of a conventional pasteurizing section at the upper end of the C2 splitter column. A bottoms product, which forms the product stream comprising ethane, propylene, and hydrocarbons heavier than propylene of step (iii) is withdrawn from the C2 splitter column.

The first liquid stream is passed to the C2 splitter with no intermediate removal of C3 components. In particular there is no deethanizer column to remove the C3 and heavier compounds from the C2 splitter feed stream. Any components heavier than ethane, such as propylene and propane enter the C2 splitter and exit in the C2 splitter bottoms stream. In step (iv) of the process of the present invention, this stream is recycled to the autothermal cracking reactor. In a conventional olefins plant such a design would be highly undesirable, since the propylene components would cause considerable coking within a conventional steam cracking furnace. An autothermal cracking reactor, on the other hand, can effectively process feeds containing propylene with no coking penalty. Propylene and other heavier olefins therefore do not need to be removed from the C2 splitter bottoms stream, reducing the number of distillation columns required and also the energy requirement of the process of this invention as compared with the prior art.

In step (v) of the process of this invention a first purified ethylene product is recovered from the first ethylene-rich product stream. The first ethylene-rich product stream typically comprises primarily, by which is meant at least 90 mol %, ethylene and is substantially free of ethane and any components lighter than ethylene. The stream may contain acetylene if acetylene is not removed from the product stream before it is chilled in step (ii). Removal of acetylene from the first ethylene-rich product stream is particularly problematic, because it consists of essentially pure ethylene with acetylene impurities. Standard back-end acetylene hydrogenation systems cannot be used on this stream because they produce ethane and heavier hydrocarbons, termed “green oils”, in the hydrogenation reactor effluent, which would require additional separation steps to remove. Instead, an acetylene removal technology is needed which does not impart significant quantities of other impurities to the otherwise pure first ethylene-rich product stream.

A preferred technology which can be used to remove acetylene from the first ethylene-rich product stream is solvent extraction. These systems utilize a solvent such as N,N-dimethylformamide (DMF), 1-methyl-2-pyrrolidone (NMP), mixtures of NMP/Methanol, or acetone to selectively absorb acetylene from a predominantly ethylene stream. With modern acetylene extraction systems, acetylene can be removed to very low levels, for example below 1 ppm by weight. Acetylene extraction systems leave relatively few contaminants in the cleaned ethylene and for this reason they can be used to remove acetylene from purified ethylene product streams, with no further purification of the ethylene being required downstream of the acetylene extraction unit.

A typical acetylene extraction unit consists of an absorber column and one or two stripping columns. A simple arrangement consisting of an absorber and a single stripping column is described in Chemical Engineering, vol 78 pp. 83-85. In this arrangement the ethylene stream containing acetylene is directed to the lower section of an absorber column where it is contacted with chilled lean solvent. The overhead of the absorber consists of a reduced-acetylene purified ethylene stream and the absorber column bottoms product consists of rich solvent. The rich solvent is directed to a stripping column where it is stripped of acetylene and other C2 components to regenerate the lean solvent.

A more complex system consisting of an absorber and two stripping columns is described in U.S. Pat. No. 3,816,976 (Stork et al.). In this arrangement the rich solvent from the absorber bottoms is directed to an ethylene stripper to recover an overhead stream consisting of ethylene and acetylene, which is then compressed and recycled to the absorber column. The bottoms from the ethylene stripper is directed to an acetylene stripper which recovers a relatively pure acetylene overhead product and a lean solvent bottoms.

Other designs and improvements have been described in the art (such as intermediate liquid injection taught in U.S. Pat. No. 4,655,798). All such designs and improvements in the acetylene extraction process are included within the scope of this invention.

In step (vi) of the process of this invention a second purified ethylene product is recovered from the second ethylene-rich product stream. The second ethylene-rich product stream typically comprises primarily, by which is meant at least 90 mol %, ethylene. The stream may contain acetylene if acetylene has not been removed in previous steps. In such a case an acetylene removal technology is needed which does not impart significant quantities of other impurities to the second ethylene-rich product stream, suitable techniques being as described above for the purification of the first ethylene-rich product stream.

In particular, the first and second ethylene-rich product streams may be combined and the combined stream treated to remove acetylene.

The invention will now be described by reference to the Figures and examples, wherein

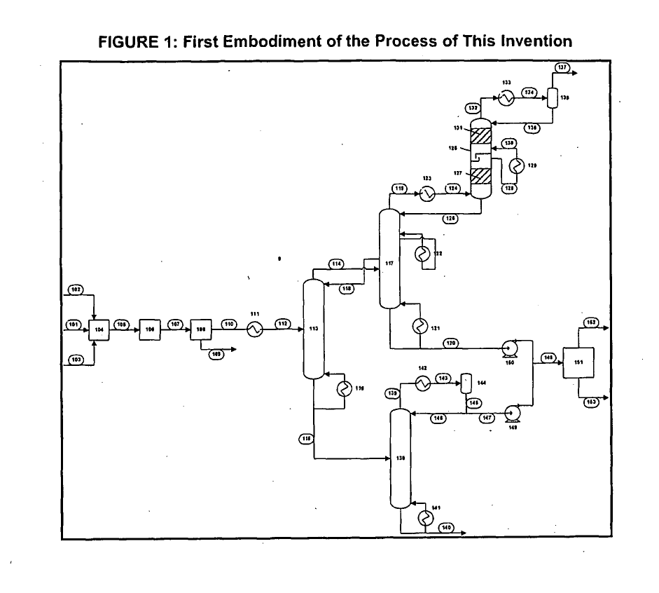

FIG. 1 depicts an embodiment of the process of this invention utilizing separate front-end ethylene distributor and demethanizer columns.

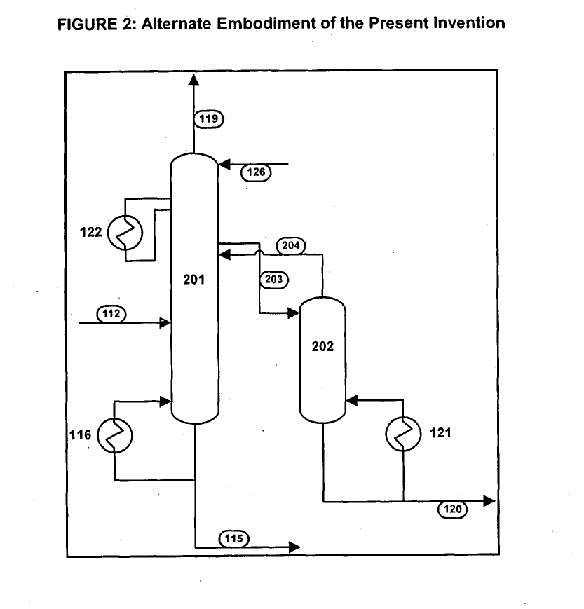

FIG. 2 depicts an alternate embodiment of the process of this invention which utilizes a front-end ethylene distributor column and side stripper.

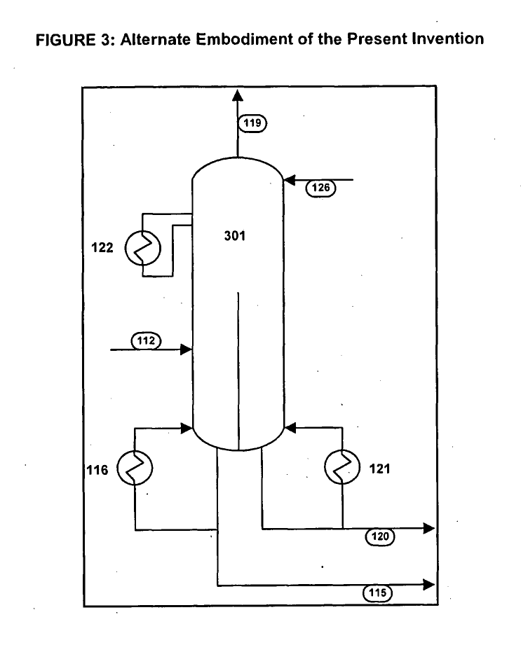

FIG. 3 depicts an alternate embodiment of the process of this invention which utilizes a front-end ethylene distributor divided wall column.

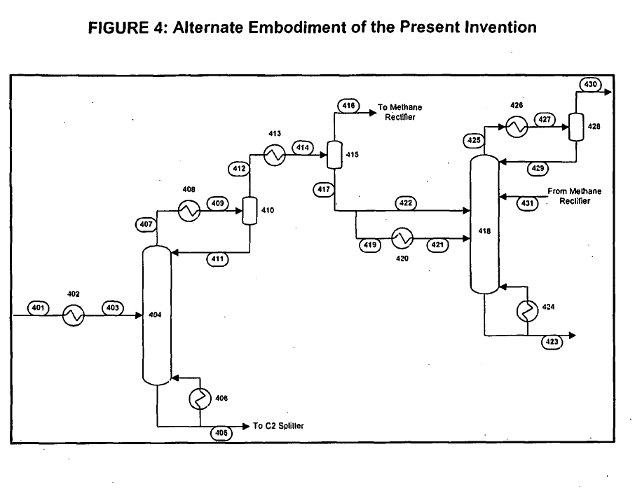

FIG. 4 depicts an alternate embodiment of the process of this invention which utilizes non-thermally coupled ethylene distributor and demethanizer columns.

It should be noted that the figures contain illustrative depictions of the invention. Some details of the process design that are well know to those skilled in the art, such as some vapor-liquid separation drums, process control valves, pumps and the like have been omitted from the drawing in order to demonstrate more clearly the key concepts of the invention. The heat exchangers shown are schematic in nature and are not meant to convey any design details or preferences.

FIG. 1 depicts an embodiment of the process of this invention that utilizes separate front-end ethylene distributor and demethanizer columns for recovery of ethylene from the effluent of an autothermal cracking reactor. In the process of FIG. 1, a hydrocarbon feed, stream 101, a hydrogen co-feed, stream 102 and a molecular oxygen-containing gas, stream 103, enter the autothermal cracking reactor 104. Stream 101 could in practice represent a number of distinct streams of different compositions, including, for example, fresh feed streams and recycle streams. Within the autothermal cracking reactor the components are reacted to form a hot effluent gas 105 which typically comprises ethylene, ethane, acetylene, propylene, methane, carbon monoxide, carbon dioxide, hydrogen, water vapor and hydrocarbons heavier than propylene. This gas is rapidly quenched by generating high-pressure steam in the quench section 106. Within the quench section the majority of the water vapor is also removed, typically through use of a water quench tower.

The cooled reactor effluent stream 107 then enters the gas conditioning section 108. In this section impurities such as carbon dioxide, residual molecular oxygen, and traces of water vapor are removed. This section typically contains an amine or caustic tower or other means for removing carbon dioxide, catalytic beds for removal of residual amounts of oxygen, and process dryers for removal of water vapor. If the autothermal reactor is operated at a relatively low pressure, for example below about 10 barg, this section would also typically contain cracked gas compressors. Optionally, all or a portion of the acetylene may be removed in this section. It should be noted that the high CO concentrations that exist in stream 107 will inhibit acetylene hydrogenation in conventional front-end acetylene hydrogenation reactors. For this reason conventional front-end acetylene hydrogenation systems may not provide sufficiently high acetylene conversions to produce product-purity ethylene later in the process.

A condensed heavy hydrocarbon stream may be generated in either section 106 or section 108, indicated by stream 109. Such a stream could be further processed as desired, recycled to the autothermal cracking reactor, or used as fuel.

The cooled, conditioned cracked gas stream 110 is chilled to around −40° C. against an external refrigerant and/or cold process streams in exchanger 111 and is directed as stream 112 to the ethylene distributor column 113. It should be noted that exchanger 111, shown as a single exchanger in FIG. 1, in practice could represent a plurality of separate exchangers. This is a significant departure from the prior art configuration of U.S. Pat. No. 5,675,054, which would direct stream 112 to a deethanizer column before entering the ethylene distributor.

The ethylene distributor column separates methane from ethane while distributing ethylene between the overhead and bottom product streams 114 and 115, respectively. Therefore the overhead stream 114 comprises methane, carbon monoxide, hydrogen, and ethylene, and is substantially free of ethane. That is, no further separation of ethane and ethylene is required in order to recover a purified ethylene product stream from stream 114. Typically the molar ratio of ethane to ethylene in stream 114 would be less than about 0.005, preferably less than about 0.001, though in some cases a molar ratio higher than 0.005 could be tolerated. If acetylene is not completely removed in the conditioning section 108, then acetylene will also be present in stream 114.

The bottoms stream 115 contains primarily ethylene, ethane, and any hydrocarbons heavier than ethane which enter the ethylene distributor column. Thus it typically contains C3 components, including propylene. If acetylene is not completely removed in the conditioning section 108, then acetylene will also be present in stream 115. In this case the acetylene could optionally be removed from stream 115 through the use of a back-end acetylene hydrogenation system (not shown), the design of which is well known to those skilled in the art. Stream 115 is substantially free of methane or lighter components, containing for example less than 0.1 mol % methane and lighter components. The column is reboiled using exchanger 116.

The overhead stream 114 enters the demethanizer column 117. The ethylene distributor is thermally coupled to the demethanizer column such that a liquid side draw stream 118 from the demethanizer provides reflux liquid to the ethylene distributor column. Stream 118 is taken from the demethanizer column at a point near that where feed stream 114 enters the column. It is directed as reflux liquid to the top of the ethylene distributor column 113. There is therefore no condenser exchanger on the ethylene distributor column.

The demethanizer column 117 serves to separate ethylene from the methane and lighter components. The overhead stream 119 therefore contains hydrogen, methane, carbon monoxide, and a low concentration of ethylene. The bottoms stream 120 contains primarily ethylene. If acetylene is not completely removed in the conditioning section 108, then acetylene will also be present in stream 120. In contrast to the prior art process of U.S. Pat. No. 5,675,054, the demethanizer column is not thermally coupled to a downstream column. Instead, stripping vapor is provided to the bottom of the demethanizer column 117 through the use of a conventional reboiler 121. The demethanizer column optionally operates with an intercondenser 122 in the enriching section, that is, above the location of feed stream 114.

Depending on the design and operation of the demethanizer column, there may be an economically recoverable amount of ethylene remaining in stream 119. FIG. 1 depicts one method for recovering additional ethylene from the demethanizer overhead vapor utilizing a combined rectification and chilling operation. In this method the demethanizer overhead stream 119 is chilled and partially condensed in exchanger 123. The resulting stream 124 is directed to the bottom of the methane rectifier column 125. The purpose of the methane rectifier column is to perform a final separation of the ethylene from the methane in stream 119. The recovered ethylene exits the methane rectifier in the bottoms stream 126, which is directed as reflux liquid to the top of the demethanizer column 117.

The methane rectifier depicted in FIG. 1 contains two rectification sections, an intercondenser and an overhead condenser. The vapor and liquid in stream 124 are separated in the bottoms section of the methane rectifier 125. The vapor travels up through the lower rectifying section 127 where it contacts downflowing liquid, thereby rectifying the upflowing vapor and recovering ethylene from it. The entire resulting rectified vapor is withdrawn from the methane rectifier column as stream 128 by means of a total vapor trap-out tray or other suitable means. Stream 128 is chilled and partially condensed in intercondenser exchanger 129, and is returned to the methane rectifier as stream 130 to a location above the total vapor trap-out tray and below the upper rectifying section 131. The vapor and liquid contained in stream 130 are separated, with the liquid providing a portion of the reflux liquid to the lower rectification section. The vapor contained in stream 130 travels upward and is further rectified in the upper rectification section.

The gross overhead vapor stream 132 from the methane rectifier is chilled and partially condensed in the overhead condenser 133 and then directed as stream 134 to the methane rectifier reflux drum 135. Liquid stream 136 from drum 135 provides reflux liquid to the upper rectification section 131. The vapor stream 137 from drum 135 is the light gas stream from the ethylene recovery system and contains primarily methane, hydrogen, and carbon monoxide. Stream 137 can also contain a small amount of residual ethylene. Typically the ethylene leaving the process in stream 137 will represent less than 5%, and preferably less than 2%, of the ethylene which enters the process in stream 112.

The light gas stream 137 can be processed further if desired. It can, for example, be expanded and then reheated to provide refrigeration to the process just described, particularly to exchangers 133, 129, 123 and 122. It should be noted that these and other exchangers in FIG. 1 could be combined into one or more multi-pass exchangers, as is well known to those skilled in the art. Optionally, all or a portion of stream 137 could be directed to a hydrogen recovery section in order to produce a purified hydrogen product. Additionally, all or a portion of it may be directed to a CO recovery section in order to produce a purified CO or synthesis gas product. All such methods of further treating the light gas stream 137 are contained within the scope of this invention.

It should be noted that other methods can be used to recover ethylene from stream 119 and thereby produce the light gas stream 137. FIG. 1 depicts a methane rectifier column 125 with two rectification sections, an intercondenser, and an overhead condenser. More or fewer rectification/condensation stages could be used. Also, the methane rectifier column 125, shown as a separate column in FIG. 1, could be combined with the demethanizer column 117 to reduce the overall capital cost of the process.

Other arrangements that combine one or more of the elements of process gas chilling, partial condensation, and rectification could also be used in place of the methane rectifier 125 in FIG. 1. For example, one or more dephlegmators, or the advanced rectification designs of U.S. Pat. Nos. 6,343,487 and 4,496,381 could be used, among others. The general guiding principle of the design of the ethylene recovery system is to ensure that stream 137 contains an economically small amount of residual ethylene. Typically, the ethylene recovery section is designed so that less than 5% and preferably less than 2% of the ethylene contained in stream 112 exits in stream 137. These and other methods that can be utilized are all contained within the scope of this invention.

The bottoms stream 115 from the ethylene distributor column 113 is directed to a C2 splitter column 138. This column separates the ethylene from the ethane and heavier components contained in stream 115 to produce an ethylene-rich overhead product stream 139 and a bottoms stream 140. The bottoms stream 140 contains ethane and any heavy hydrocarbons contained in stream 115, including propylene. Stream 140 contains little if any ethylene and is recycled to the autothermal cracking reactor 104. Column 138 is reboiled using reboiler 141.

Stream 115 enters column 138 with no intermediate removal of C3 components. In particular there is no deethanizer column to remove the C3 and heavier compounds from the C2 splitter feed stream. Therefore any components heavier than ethane, such as propylene and propane, contained in stream 115 enter the C2 splitter and exit in the C2 splitter bottoms stream.

The overhead vapor stream 139 is condensed with condenser exchanger 142 and the resulting stream 143 is directed to the C2 splitter reflux drum 144. The liquid stream 145 from drum 144 is split into two streams. Stream 146 is directed as reflux liquid to the C2 splitter and stream 147 is withdrawn as the net ethylene-rich product from the column. Stream 147 contains primarily ethylene and may also contain acetylene. If desired, condenser 142 can be operated as a partial condenser and the net ethylene-rich product stream 147 would be taken as a vapor from drum 144.

Stream 147 is combined with stream 120 to form a combined ethylene stream 148. If desired, these streams may be pumped with pumps 149 and 150 as shown. If sufficient acetylene was removed from the autothermal cracking reactor effluent in the conditioning section 108, then stream 148 represents the final purified ethylene product from the process. If insufficient removal of acetylene was accomplished in the conditioning section 108, then stream 148 may be treated in an acetylene removal process 151. The acetylene removal process 151 produces a purified ethylene stream 152 and optionally an acetylene-rich stream 153. The purified ethylene stream 152 is withdrawn as the final purified ethylene product.

The acetylene removal process 151 can be an acetylene extraction process in which acetylene is preferentially absorbed into a liquid solvent, or it may be any other suitable process which does not impart significant levels of undesirable impurities to the ethylene product stream 152. A conventional back-end hydrogenation system therefore would not be a preferred method to remove acetylene from either stream 120 or the combined stream 148 because it generates significant quantities of ethane and heavier hydrocarbons which would remain in the ethylene product stream 152.

The acetylene-rich stream 153 can be treated in a variety of ways. A purified acetylene product can be recovered from it as is well known in the art. The acetylene contained in this stream may be partially or completely hydrogenated to ethylene and/or ethane, and the resulting stream recycled to a location within the process of FIG. 1. For example, such a hydrogenated stream can enter the C2 splitter column 138 for separation of the ethylene and ethane contained therein. If the conditioning section 108 contains a partial acetylene removal step, then stream 153 may be recycled to section 108. These and all other options for treating the acetylene-rich stream 153 are contained within the scope of this invention.

Those skilled in the art will recognize that the combination of the thermally-coupled ethylene distributor column 113 and demethanizer column 117 can be implemented in a number of ways. In addition to the separate thermally coupled columns shown in FIG. 1, other alternative implementations are possible:

FIG. 2 depicts one alternative implementation of these columns which consists of a main ethylene distributor column and thermally-coupled side stripper. In this implementation the ethylene distributor 113 of FIG. 1 is combined with the enriching section of the demethanizer column 117 to form a main ethylene distributor column 201. The sidestripper column 202 serves the function of the demethanizer stripper section of FIG. 1. Other components such as reboilers and intercondensers serve the same function in both FIG. 1 and FIG. 2 and therefore have identical numbers in both Figures. Some of the streams in FIG. 2 have essentially the same composition and flow as those in FIG. 1 and therefore have identical numbers in both Figures.

Stream 112 enters the main ethylene distributor column 201 at an intermediate location, stream 119 is withdrawn from the top of the main ethylene distributor column, and steam 115 is withdrawn from the bottom of the main ethylene distributor column. A liquid sidestream 203 is withdrawn from the main ethylene distributor column at a point between where stream 112 enters and stream 119 exits, and where the liquid within the column is substantially free of ethane, for example, where the molar ratio of ethane to ethylene is less than about 0.005, preferably less than 0.001. The liquid sidedraw 203 therefore contains primarily ethylene along some methane and other light gases. It is directed to the top of a sidestripper column 202 in which the light gases are removed from the liquid. Stream 120 is withdrawn as the sidestripper bottoms. The sidestripper overhead stream 204 contains light gases and some ethylene and is directed back to the main ethylene distributor column 201, to a point near where stream 203 was taken.

FIG. 3 depicts another alternate embodiment of the process of this invention which utilizes a front-end ethylene distributor divided wall column. This embodiment combines the separation functions of the main ethylene distributor 201 and sidestripper column 202 described hereinabove and depicted in FIG. 2 into a single ethylene distributor divided wall column 301. This implementation is similar in design but not in function to an implementation taught in published U.S. patent application US 20044182751. Some components such as reboilers and intercondensers serve the same function in both FIG. 2 and FIG. 3 and therefore have identical numbers in both Figures. Some of the streams in FIG. 3 have essentially the same composition and flow as those in FIG. 2 and therefore have identical numbers in both Figures.

In this case the dividing wall exists in a lower portion of the column and extends within the column from an intermediate point all the way to the bottom of the column. The dividing wall thereby provides for a single rectification section above the wall and for two separate half-sections in the lower portion of the column and on either side of the wall. The feed stream 112 enters one of the half-sections at a point below the top of the dividing wall. The section which the feed enters, along with the upper rectification section, serve the same function as the main ethylene distributor column 201 of FIG. 2. The other half-section functions as the sidestripper column 202 of FIG. 2. Stream 119 is withdrawn from the top of the ethylene distributor divided wall column, stream 115 is withdrawn from the bottom of the ethylene distributor section, and stream 120 is withdrawn from the bottom of the sidestripper half-section.

FIG. 4 depicts another alternate embodiment of the process of this invention which utilizes non thermally coupled ethylene distributor and demethanizer columns. In all of the previous embodiments the ethylene distributor and demethanizer functions were thermally coupled. In other words, in each of the embodiments at least a portion of the reflux liquid for the ethylene distributor (or to the ethylene distributor sections of the main column of FIG. 2 or the divided wall column of FIG. 3) was derived from a liquid side draw from the demethanizer column (or from the demethanizer sections of the main column of FIG. 2 or the divided wall column of FIG. 3).

We have determined that such thermal coupling, while beneficial from an energy standpoint, is not critical to the operation of this invention. In the alternate embodiment of FIG. 4 reflux liquid for the ethylene distributor column is derived through a conventional partial condenser arrangement. In FIG. 4 the gas stream 401 from the conditioning section (not shown) is chilled in exchanger 402 to about −40° C. and directed as stream 403 to the front-end ethylene distributor column 404. The bottoms from the ethylene distributor are withdrawn as stream 405 and contain ethylene, ethane, and components heavier than ethane. Stream 405 is directed to a C2 splitter column (not shown) to recover a purified ethylene product from it. Stripping vapor is supplied to the base of the ethylene distributor using a conventional reboiler 406.

The gross overhead vapor is withdrawn from the ethylene distributor as stream 407 and is chilled and partially condensed in exchanger 408. The vapor and liquid in the resulting stream 409 are separated in reflux drum 410, and the liquid withdrawn and directed as reflux stream 411 to the top of the ethylene distributor column. The vapor stream 412 from drum 410 contains ethylene, methane, carbon monoxide and hydrogen. It is further chilled and partially condensed in exchanger 413. The vapor and liquid in stream 414 are separated in demethanizer feed drum 415. The vapor stream 416 from drum 415 contains methane, carbon monoxide and hydrogen and some ethylene. It can be directed to a methane rectifier similar in principle to column 125 in FIG. 1 if desired. If no further ethylene recovery is desired stream 416 could be directed to a hydrogen recovery and/or a CO recovery section.

The liquid stream 417 from drum 415 is fed to the demethanizer column 418. The demethanizer makes a sharp split between ethylene and components lighter than ethylene. If desired the split feed arrangement shown in FIG. 4 can be used. In this design stream 417 is split into two streams. Stream 419 is at least partially vaporized in exchanger 420 and is directed as stream 421 to the demethanizer column 418. Stream 422 constitutes the remainder of stream 417 and is directed to the demethanizer 418. If the demethanizer column operates at a pressure significantly less than that of drum 415, valves or other pressure reducing devices can be place on streams 419 and 422. The bottoms stream 423 from the demethanizer contains a purified ethylene product. Stripping vapor is provided to the demethanizer through the use of a conventional reboiler 424.

The gross overhead vapor stream 425 from the demethanizer is chilled and partially condensed in the demethanizer condenser 426. The vapor and liquid in the resulting stream 427 are separated in the demethanizer reflux drum 428 and the liquid returned as reflux stream 429 to the demethanizer tower. The vapor stream 430 from drum 428 contains primarily methane, carbon monoxide and some hydrogen. It can be directed to a CO recovery section if desired or used as fuel. If a methane rectifier (not shown) is used, the bottoms stream 431 from the methane rectifier would enter the demethanizer as shown.

Note that exchangers 408, and 413, and 426 require refrigeration duty. It may be possible in practice to combine two or more of these exchangers into a single multipass cryogenic exchanger, as is well known to those skilled in the art. Many sources of refrigeration could be used, but we have found it beneficial to provide chilling duty to 408, 413, and optionally 426 through a combination of a mixed refrigeration system and the reheating of cold process gases.

The use of mixed refrigeration systems in the production of olefins is commercially practiced. In a mixed refrigerant system the working fluid is composed of more than one component. For example, the working fluid can be composed of a mixture of hydrocarbons such as methane, ethane, ethylene, propane, propylene, butane, or butene. In a closed-loop mixed refrigerant system, the working fluid is first compressed and then cooled and condensed. The mixed refrigerant liquid is then flashed to lower pressure whereupon any remaining liquid is vaporized to provide chilling duty to the process at a temperatures below ambient temperature. The relatively low-pressure vaporized mixed refrigerant working fluid is then recycled to the compressor to be re-compressed and start the cycle over again.

All of the possible column design options presented hereinabove for implementing the separations carried out by columns 113 and 117 of FIG. 1 to produce stream 115, 119, and 120 are contained within the scope of this invention.

### EXAMPLE

An example of an embodiment of this invention was simulated using a commercially available simulation package. The process simulated in the example is identical to the preferred embodiment of FIG. 1. The feed to the autothermal cracking reactor in this example is pure ethane and the unit was sized to produce approximately 800 KTA of ethylene. The autothermal cracking reactor is operated at 30 barg and there was no further compression of the cracked gas. The net refrigeration requirements were met by utilizing a cascaded propylene/mixed refrigerant system. Stream 110 enters exchanger 111 at 15° C. The acetylene-rich stream 153 in FIG. 1 was assumed to be hydrogenated to a mixture of ethylene and ethane and recycled to the C2 splitter column 138. The flow rates and compositions of key streams in FIG. 1 are given in Table 1, and the key heat exchanger duties are given in Table 2.

Note that this example embodies the main elements of the process of this invention. Namely, the entire cracked gas enters the ethylene distributor column 113 without any separation of the C3 components. Also, the ethylene distributor bottoms stream 115 directly enters the C2 splitter column 138 without any intermediate separation of C3 components. The C2 splitter bottoms stream, therefore contains essentially all of the C3 and heavier components that exist in the cracked gas. This stream is recycled to the autothermal cracking reactor. Finally, the light gas stream 137 contains less than 5% of the ethylene contained in the cracked gas stream.

The total refrigeration energy requirement for the separation process of this example is 29.60 MW. This is made up of the propylene refrigeration compressor power (16.60 MW), the mixed refrigeration compressor power (11.33 MW) and miscellaneous compressors (1.67 MW).

When a front-end partial deethanizer similar in concept to that of U.S. Pat. No. 5,675,054 was added to the flowsheet, the total refrigeration energy of the resulting prior art process is higher at 31.28 MW. This total is made up of the propylene refrigeration compressor (17.14 MW), the mixed refrigeration compressor (12.47 MW) and miscellaneous compressors (1.67 MW).

Additional energy savings over the process of U.S. Pat. No. 5,675,054 are expected, because the ethylene distributor in the present invention utilizes a conventional reboiler (121 in FIG. 1), whereas in the process of U.S. Pat. No. 5,675,054 the ethylene distributor bottom is thermally coupled to a downstream column. As described in US2004/182752, operation of an ethylene distributor with a conventional reboiler requires less energy than a process with a thermally-coupled ethylene distributor, when the process is coupled with a conventional vapor recompression refrigeration system.

In addition, the prior art process of U.S. Pat. No. 5,675,054 includes one additional column than the process of this invention (a deethanizer column), and so it will likely have a higher capital cost. It is clear from this comparison that the process of this invention, which utilizes a front-end ethylene distributor rather than the prior-art front-end deethanizer column, is more energy-efficient than the prior art process.

TABLE 1
Flows and Conditions for Streams in the Example (FIG. 1)

| | Stream No. | | | | | | | | |
|---|---|---|---|---|---|---|---|---|---|
| | 112 | 114 | 115 | 119 | 120 | 126 | 132 | 137 | 140 |
| Temperature (Deg C.) | −40.0 | −52.4 | −3.6 | −107.0 | −20.2 | −107.6 | −128.4 | −137.0 | −8.3 |
| Pressure (barg) | 25 | 24 | 25 | 24 | 24 | 22 | 22 | 22 | 16 |
| Vapor Fraction | 0.82 | 1.00 | 0.00 | 1.00 | 0.00 | 0.00 | 0.00 | 1.00 | 0.00 |
| Molar flows (kg mol/hr) | | | | | | | | | |
| HYDROGEN | 3524.0 | 3576.1 | 0.0 | 3528.1 | 0.0 | 4.2 | 3532.0 | 3524.0 | 0.0 |
| CO | 1618.3 | 1696.7 | 0.0 | 1635.9 | 0.0 | 17.6 | 1668.4 | 1618.3 | 0.0 |
| METHANE | 1681.9 | 1944.7 | 0.4 | 1773.0 | 0.2 | 91.8 | 2015.0 | 1681.2 | 0.0 |
| ETHYLENE | 3452.5 | 6173.7 | 2206.4 | 310.5 | 1236.5 | 300.9 | 50.5 | 9.6 | 17.8 |
| ETHANE | 3011.6 | 1.9 | 3011.3 | 0.0 | 0.4 | 0.0 | 0.0 | 0.0 | 3025. |
| ACETYLENE | 32.6 | 23.1 | 27.9 | 0.5 | 4.6 | 0.5 | 0.0 | 0.0 | 17.4 |
| PROPYLENE | 217.3 | 0.0 | 217.3 | 0.0 | 0.0 | 0.0 | 0.0 | 0.0 | 217.3 |
| PROPANE | 47.1 | 0.0 | 47.1 | 0.0 | 0.0 | 0.0 | 0.0 | 0.0 | 47.1 |
| PROPDIENE | 5.2 | 0.0 | 5.2 | 0.0 | 0.0 | 0.0 | 0.0 | 0.0 | 5.2 |
| ISOBUTANE | 29.0 | 0.0 | 29.0 | 0.0 | 0.0 | 0.0 | 0.0 | 0.0 | 29.0 |
| C4+ | 227.8 | 0.0 | 227.8 | 0.0 | 0.0 | 0.0 | 0.0 | 0.0 | 227.8 |

TABLE 2
Heat Exchanger Duties for the Example (FIG. 1)

| Exchanger | Net Duty (MW) |
|---|---|
| 111 | −16.87 |
| 116 | 8.47 |
| 122 | −16.19 |
| 123 | −4.37 |
| 129 | −1.62 |
| 133 | −1.38 |
| 141 | 33.09 |
| 142 | −37.40 |

What is claimed is:

1. A process for the production of ethylene from a paraffinic hydrocarbon-containing feed, said process comprising the steps of:
(i) autothermally cracking the paraffinic hydrocarbon-containing feed with a molecular oxygen-containing gas in contact with a catalyst capable of supporting combustion beyond the normal fuel rich limit of flammability to produce a product stream comprising ethylene, ethane, propylene, methane, carbon monoxide, hydrogen and hydrocarbons heavier than propylene,
(ii) chilling at least a portion of said product stream and, without first passing said chilled product stream through a deethanizer, passing the chilled product stream to a first separations step comprising one or more distillation columns, one of which operates as an ethylene distributor column, to recover a light gas stream comprising methane, carbon monoxide, hydrogen and less than 5% of the ethylene contained in said chilled product stream, a first liquid stream comprising ethylene, ethane, propylene and hydrocarbons heavier than propylene and substantially free of components lighter than ethylene, and a first ethylene-rich product stream,
(iii) directing said first liquid stream to a second separations step comprising a C2 splitter distillation column, to recover a second ethylene-rich product stream, a product stream comprising ethane, propylene, and hydrocarbons heavier than propylene,
(iv) recycling said product stream comprising ethane, propylene, and hydrocarbons heavier than propylene to the autothermal cracking reactor of step (i),
(v) recovering from said first ethylene-rich product stream a first purified ethylene product, and
(vi) recovering from said second ethylene-rich product stream a second purified ethylene product.

2. The process of claim 1 wherein step (ii) comprises the steps of:
(a1) directing the chilled product stream to an ethylene distributor distillation column and recovering therefrom an ethylene distributor overhead stream comprising ethylene, methane, carbon monoxide, and hydrogen and substantially free of ethane, and an ethylene distributor bottoms stream comprising ethylene, ethane, propylene, and optionally components heavier than propylene and substantially free of components lighter than ethylene;
(b1) withdrawing the ethylene distributor bottoms stream as the first liquid stream of step (ii);
(c1) directing at least a portion of the ethylene distributor overhead stream to a demethanizer column and recovering therefrom a demethanizer overhead stream comprising methane, carbon monoxide and hydrogen and a demethanizer bottoms stream comprising ethylene and substantially free of components lighter than ethylene;
(d1)) recovering said light gas stream of step (ii) from the demethanizer overhead stream of step (c1); and
(e1) withdrawing the demethanizer bottoms stream as the first ethylene rich product stream of step (ii).

3. The process of claim 2 wherein reflux liquid for the ethylene distributor column is provided by a liquid sidedraw stream from the demethanizer column.

4. The process of claim 2 wherein reflux liquid for the ethylene distributor column is provided through partial condensation of the gross ethylene distributor overhead vapor stream.

5. The process of claim 4 wherein chilling duty for said partial condensation is provided by a closed-loop mixed refrigeration system.

6. The process of claim 1 wherein step (ii) comprises the steps of:
(a2) directing the entire chilled product stream to an ethylene distributor distillation column and recovering therefrom an ethylene distributor column overhead stream comprising methane, carbon monoxide, hydrogen and less than 5% of the ethylene contained in said chilled product stream, and an ethylene distributor column bottoms stream comprising ethylene, ethane, propylene, and hydrocarbons heavier than propylene and substantially free of components lighter than ethylene;
(b2) recovering said light gas stream of step (ii) from the ethylene distributor column overhead stream of step (a2);
(c2) withdrawing the ethylene distributor column bottoms stream as the first liquid stream of step (ii);
(d2) withdrawing a liquid sidedraw stream from the ethylene distributor column at a point intermediate between the top of the ethylene distributor column and the point where the chilled product stream enters the ethylene distributor column, wherein the liquid sidedraw stream comprises ethylene and methane, and is substantially free of ethane;
(e2) directing the liquid sidedraw stream to the top of a sidestripper column and recovering therefrom a sidestripper bottoms stream comprising ethylene and substantially free of components lighter than ethylene, and a sidestripper overhead stream comprising methane;
(f2) withdrawing the sidestripper bottoms stream as the first ethylene-rich product stream of step (ii), and
(g2) directing the sidestripper overhead stream to the ethylene distributor column of step (a2).

7. The process of claim 6 wherein the ethylene distributor column is a divided wall column performing the functions of the both an ethylene distributor column and the sidestripper column.

8. The process of claim 7 wherein the ethylene distributor divided wall column produces an overhead stream comprising primarily methane, carbon monoxide and hydrogen, and forms the light gas stream of step (ii), a first bottoms product which forms the first liquid stream of step (ii) and a second bottoms product which forms the first ethylene-rich product stream of step (ii).

9. The process of claim 2 in which the ethylene distributor column is reboiled using a conventional reboiler exchanger.

10. The process of claim 2 in which both the ethylene distributor column and the demethanizer column are reboiled using conventional reboiler exchangers.

11. The process of claim 6 in which both the main ethylene distributor column and the sidestripper column are reboiled using conventional reboiler exchangers.

12. The process of claim 7 in which the ethylene distributor divided wall column employs at least two conventional reboilers, at least one arranged on each side of the dividing wall.

13. The process of claim 1 wherein at least a portion of any acetylene in the product stream of step (i) is removed from said product stream prior to step (ii).

14. The process of claim 1 wherein any acetylene in the first liquid stream is removed from said stream before it is directed to the second separations step.

15. The process of claim 1 wherein the first ethylene-rich product stream of step (ii) comprises acetylene and wherein step (v) comprises removing acetylene from said stream.

16. The process of claim 15 wherein the removal of acetylene from said first ethylene-rich product stream is carried through the use of a solvent extraction process.

17. The process of claim 1 wherein the second ethylene-rich stream of step (iii) comprises acetylene and wherein step (vi) comprises removing acetylene from said stream.

18. The process of claim 1 wherein acetylene is removed from either or both of the first purified ethylene product of step (v) and the second purified ethylene product of step (vi) through the use of a solvent extraction process.
